(19) United States
(12) Patent Application Publication
(10) Pub. No.: US 2018/0008841 A1
(43) Pub. Date: Jan. 11, 2018

(54) THERAPY PLANNING APPARATUS AND PARTICLE RADIATION THERAPY APPARATUS

(71) Applicant: MITSUBISHI ELECTRIC CORPORATION, Chiyoda-ku, Tokyo (JP)

(72) Inventors: Takaaki IWATA, Tokyo (JP); Hisashi HARADA, Tokyo (JP)

(73) Assignee: MITSUBISHI ELECTRIC CORPORATION, Chiyoda-ku, Tokyo (JP)

(21) Appl. No.: 15/545,123

(22) PCT Filed: Apr. 9, 2015
(86) PCT No.: PCT/JP2015/061085

§ 371 Date: Jul. 20, 2017

(51) Int. Cl. A61N 5/10

(52) U.S. Cl. CPC A61N 5/103; A61N 5/1081; A61N 5/1043; A61N 2005/1087

(57) ABSTRACT

A treatment planning apparatus includes an overall data management unit for storing a target irradiation dose distribution to be formed in an irradiation object, a broad irradiation parameter calculation unit and a scanning irradiation parameter calculation unit for cooperatively calculating and determining operational parameters for devices, such as an accelerator and an irradiation nozzle, to operate during a broad irradiation and an scanning irradiation, respectively, so that the sum of irradiation doses imparted by both broad irradiation and scanning irradiation forms the target irradiation dose distribution.

### TECHNICAL FIELD

The present invention relates to a particle beam therapy system that performs particle beam irradiation for cancer treatment and the like as application of a particle beam, and more particularly to a treatment planning apparatus for the therapy system.

### BACKGROUND ART

Particle beam irradiation methods for particle beam therapy systems are roughly categorized into two methods: a broad irradiation method and a scanning irradiation method. In a wobbler method, which is one type of the broad irradiation method, a charged particle beam is spread by being scanned with scanning electromagnets in a circular pattern and is shaped to irradiate an irradiation object in accordance with the shape thereof. The scanning irradiation method is for performing irradiation by scanning a charged particle beam across an irradiation object with scanning electromagnets. In a scanning irradiation method, irradiation is generally performed with the irradiation dose being controlled for each of irradiation points. In the broad irradiation method typified by the wobbler method, on the other hand, irradiation is performed with the irradiation dose being not controlled for each of irradiation points but controlled for an irradiation region as a whole.

The wobbler method is a conventionally used irradiation method, and has a merit in that there have been many actual results in clinical practice but has a demerit in that a bolus (typically formed of resin) needs to be fabricated to mimic a distal shape of a diseased site on a patient-by-patient basis.

The scanning irradiation method, although having a merit of performing a three-dimensional irradiation with increased flexibility, has some demerits such as in that there have yet been fewer actual results in clinical practice than the broad irradiation method because the scanning irradiation method is a recent technology and in that optimization calculation takes time to formulate a treatment plan.

From a viewpoint of development of irradiation apparatuses in particle beam therapy systems, an irradiation nozzle for the wobbler method was first developed and then an irradiation nozzle for the scanning irradiation method was developed. Buyers of those days were requested to alternatively decide, as an irradiation apparatus in one treatment room, whether an irradiation nozzle for the broad irradiation method or an irradiation nozzle for the scanning irradiation method. After that, there were proposed an irradiation nozzle that achieved both broad irradiation and scanning irradiation with one irradiation nozzle, and there were also proposed a gantry that was provided with two irradiation lines (Patent Document 1), thus increasing flexibility in the treatment methods.

In an irradiation nozzle provided to one irradiation line, there is also known an irradiation nozzle that operates in either one of a broad irradiation configuration and a scanning irradiation configuration and retracts the other unused configuration not to interrupt the charged particle beam irradiation (Patent Documents 2 and 3).

### PRIOR ART DOCUMENT

### Patent Documents

Patent Document 1: JP2010-158479 A;

Patent Document 2: JP2009-236867 A;

Patent Document 3: WO2013/011583 A1

### SUMMARY OF THE INVENTION

### Problem that the Invention is to Solve

While a gantry equipped with two irradiation lines and an irradiation nozzle capable of performing the broad irradiation and the scanning irradiation with one irradiation nozzle have been thus put into practical use, usage thereof is no more than that one of the irradiation methods is alternatively determined for one patient. This poses a problem in that the demerit of the broad irradiation method still remains when the broad irradiation method is selected or the demerit of the scanning irradiation method still remains when the scanning irradiation method is selected.

In consideration of the above problem, the present invention is aimed at achieving a highly accurate and highly flexible irradiation that utilizes the respective merits of the broad irradiation and the scanning irradiation by performing these irradiations for one diseased site.

### Means for Solving the Problem

The present invention offers a treatment planning apparatus configured to formulate a treatment plan that allows a particle beam therapy system to irradiate an irradiation object with a particle beam extracted from an accelerator, by switching between a scanning irradiation from a scanning irradiation nozzle mounted with scanning irradiation use parts for the scanning irradiation that is performed while shifting the particle beam and controlling an irradiation dose imparted to each of points in the irradiation object and a broad irradiation from a broad irradiation nozzle mounted with broad irradiation use parts for the broad irradiation that is performed by controlling a total irradiation dose imparted to a region in the irradiation object, the treatment planning apparatus includes an overall data management unit configured to store a target irradiation dose distribution to be formed in the irradiation object; a broad irradiation parameter calculation unit configured to calculate operational parameters for respective devices, such as the accelerator and the broad irradiation nozzle, to operate during the broad irradiation; and a scanning irradiation parameter calculation unit configured to calculate operational parameters for the respective devices, such as the accelerator and the scanning irradiation nozzle, to operate during the scanning irradiation, wherein the broad irradiation parameter calculation unit and the scanning irradiation parameter calculation unit cooperatively calculate and determine the operational parameters for the respective devices, such as the accelerator and the broad irradiation nozzle, to operate during the broad irradiation and the operational parameters for the respective devices, such as the accelerator and the scanning irradiation nozzle, to operate during the scanning irradiation, so that the sum of irradiation doses imparted by both broad irradiation and scanning irradiation forms the target irradiation dose distribution.

### Advantage of the Invention

A treatment planning apparatus according to the present invention enables a treatment plan to be formulated in a short time and allows for constructing a particle beam therapy system that imparts irradiation doses with high accuracy to diseased sites of various shapes.

### BRIEF DESCRIPTION OF THE DRAWINGS

FIG. 1 is a block diagram showing an overall configuration of a particle beam therapy system including a treatment planning apparatus according to Embodiment 1 of the present invention;

FIG. 2 is a side view showing a scanning irradiation use parts mounting configuration of an example of an irradiation nozzle equipped in the particle beam therapy treatment system to which the treatment planning apparatus of the present invention is applied;

FIG. 3 is a side view showing a broad irradiation use parts mounting configuration of the example of the irradiation nozzle equipped in the particle beam therapy system to which the treatment planning apparatus of the present invention is applied;

FIG. 4 is a schematic diagram showing an example of an irradiation apparatus equipped with two irradiation lines that is installed in the particle beam therapy system to which the treatment planning apparatus of the present invention is applied;

FIGS. 5A and 5B show schematic illustrations of an irradiation based on a treatment plan formulated by the treatment planning apparatus according to Embodiment 1 of the present invention;

FIG. 6 is a flow diagram showing an operation flow of the treatment planning apparatus according to Embodiment 1 of the present invention;

FIG. 7 shows a schematic illustration of an irradiation based on a treatment plan formulated by the treatment planning apparatus according to Embodiment 2 of the present invention; and

FIG. 8 shows a schematic illustration of an irradiation based on a treatment plan formulated by the treatment planning apparatus according to Embodiment 3 of the present invention.

### EMBODIMENTS FOR CARRYING OUT THE INVENTION

### Embodiment 1

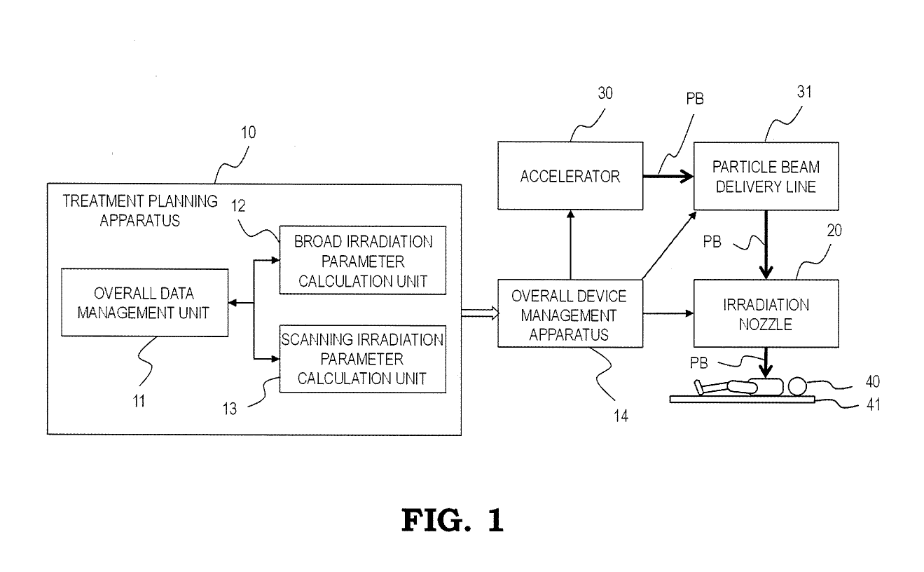

FIG. 1 is a block diagram showing an overall configuration of a particle beam therapy system including a treatment planning apparatus according to Embodiment 1 of the present invention. A particle beam PB extracted from an accelerator 30 is guided to an irradiation nozzle 20 through a particle beam delivery line 31. The irradiation nozzle 20 is provided with various parts and configured to switch between a broad irradiation and a scanning irradiation to irradiate with the particle beam PB a diseased site of a patient 40, an irradiation object. Meanwhile, a treatment plan how to irradiate the diseased site with the particle beam is in advance formulated appropriately to the diseased site of the patient by a treatment planning apparatus 10. The treatment planning apparatus 10 determines and stored therein operational parameters for respective devices, such as the accelerator 30, the particle beam delivery lines 31, and the irradiation nozzle 20, of the particle beam therapy system for it to perform irradiation in accordance with the formulated treatment plan. The operational parameters are sent from, for example, an overall device management apparatus 14 to the respective devices for them to be controlled so as to operate in accordance with the treatment plan during treatment, i.e., during irradiating the patient diseased site with the particle beam.

FIG. 1 also shows a schematic configuration of the treatment planning apparatus 10 according to the present invention. An overall data management unit 11 stores therein irradiation distribution data for the patient diseased site to be irradiated. The data contains, for example, a three-dimensional irradiation region and an irradiation dose distribution in the irradiation region. The irradiation dose distribution is referred here to as a target irradiation dose distribution. The treatment planning apparatus 10 is further has a broad irradiation parameter calculation unit 12 and a scanning irradiation parameter calculation unit 13. The broad irradiation parameter calculation unit 12 calculates parameters for the respective devices to perform irradiation using a broad irradiation method (hereinafter, referred to as broad irradiation), and the scanning irradiation parameter calculation unit 13 calculates parameters for the respective devices to perform irradiation using a scanning irradiation method (hereinafter, referred to as scanning irradiation). The treatment planning apparatus of the present invention formulates the treatment plan so that the target irradiation dose distribution is formed by combination of the broad irradiation and the scanning irradiation, as will be described later. The operational parameters for the respective devices thus calculated according to the treatment plan is stored, for example, in the overall data management unit 11, and the operational parameters stored are sent from, for example, an overall device management apparatus 14 to the respective devices for them to operate in accordance with the parameters during treatment.

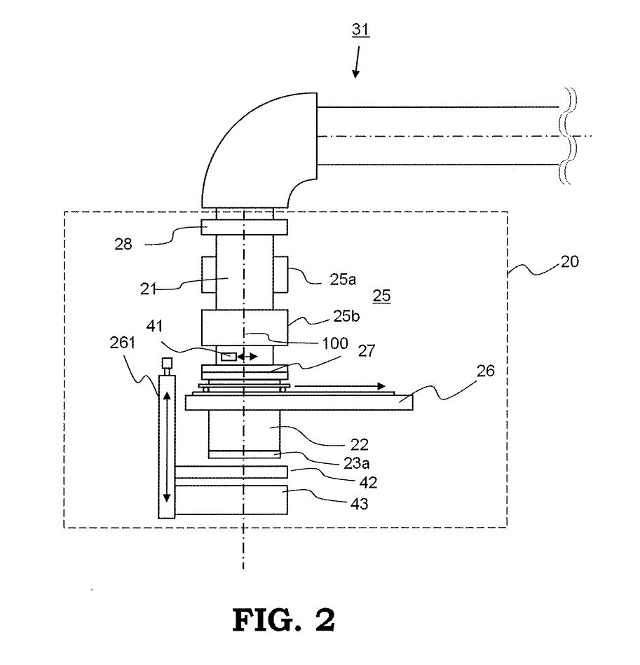

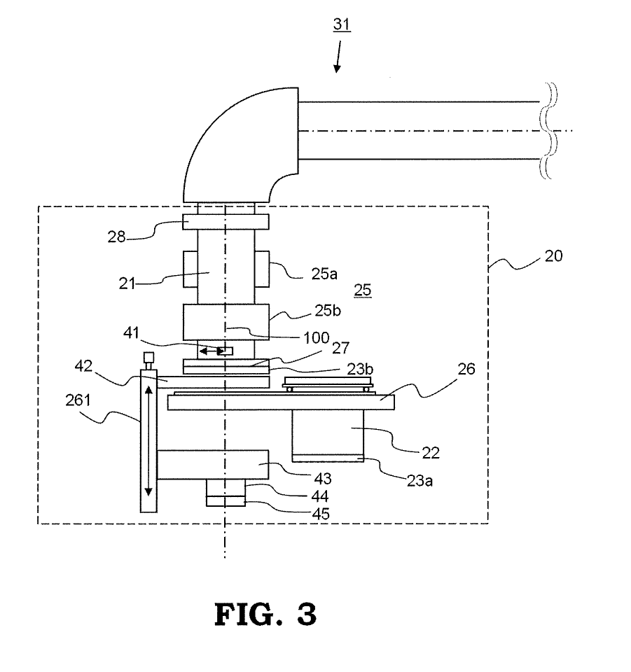

FIGS. 2 and 3 show examples of configurations of the irradiation nozzle capable of the scanning irradiation and the broad irradiation by switching therebetween. FIG. 2 shows an example of a scanning irradiation configuration of the irradiation nozzle. This configuration is disclosed, for example, in Patent Document 3. As shown in FIG. 2, the particle beam extracted from the accelerator 30 is delivered to the irradiation nozzle 20 through the particle beam delivery line 31 configured with vacuum ducts and deflectors; then passes through a connecting vacuum duct 21 and a scanning irradiation use vacuum duct 22 provided in the irradiation nozzle 20 for keeping the vacuum condition; and is extracted to the atmosphere from a beam extraction window 23a to irradiate the diseased site, the irradiation object. The particle beam is scanned across the irradiation object by scanning electromagnets 25a and 25b (also referred to as scanning electromagnets 25 collectively). The irradiation nozzle 20 is further provided with a vacuum duct moving mechanism 26 for retracting the scanning irradiation use vacuum duct 22 from the beam line 100, which is the beam center line indicated by the dot-dash line, to switch the parts arrangement to that for the broad irradiation; a connection flange plane 27 of the vacuum duct 21; a gate valve 28 for separating the vacuum condition in the vacuum duct 21 at a position immediately upstream the scanning electromagnets 25; and a scatterer 41 for scattering the particle beam in conformity to the irradiation region. The irradiation nozzle is further provided with a ridge filter 42 for spreading out the Bragg peak of the particle beam in the depth-wise direction and a range shifter 43 for adjusting the penetration range of the particle beam. The ridge filter 42 and the range shifter 43 here are attached to a ridge filter moving mechanism 261 so as to be movable in the direction parallel to the beam line 100.

Next, the operation of the irradiation nozzle will be described. In a case of the scanning irradiation, in order to make small the beam spot size at beam irradiation points by suppressing scattering of the particle beam as far as possible, a vacuum duct is generally disposed close up to the beam irradiation points. Since the scatterer 41 is unnecessary in this case, it is retracted to the side of the beam line 100 within the vacuum duct 21. In a case of the particle beam being a proton beam, the ridge filter 42 is unnecessary for the scanning irradiation; however, a ridge filter may be used in some cases for the other particle beam, to increase the energy width slightly. For example, in a case of a heavy particle beam such as a carbon ion beam, since the beam has a very sharp Bragg peak width compared to a proton beam, a ridge filter may be used to form a spread-out Bragg peak (SOBP) in order to irradiate a certain depth width (several mm) in one scan thereby to reduce irradiation time. Note that the ridge filter is for spreading out the Bragg peak width to several mm and its bar ridge height may be shorter than the SOBP width even though the ridge filter is disposed at a position not away from the irradiation object. Accordingly, a ridge filter can be used for the heavy particle beam that is manufactured much easier than that for the broad irradiation. Furthermore, the penetration depth (penetration range) of the particle beam depends on the energy of the particle beam; hence, varying the energy of the particle beam is necessary to vary the penetration range thereof. Performing change of the energy only by energy adjustment of the accelerator poses a problem of taking time to change the energy. For that reason, a range shifter for reducing the energy of the particle beam is used in some cases to vary the energy of the particle beam. Considering the fact that the particle beam is scattered by the range shifter, the range shifter is desirably disposed as downstream as possible, in other words, in a position as close as possible to the irradiation object. Accordingly, in performing the scanning irradiation using the ridge filter 42 and the range shifter 43, they are preferably arranged as shown in FIG. 2.

Switching from the scanning irradiation to the broad irradiation is described next. FIG. 3 shows a broad irradiation configuration of the irradiation nozzle 20 switched from the scanning irradiation configuration shown in FIG. 2. In the scanning irradiation, the scanning irradiation use vacuum duct 22 is disposed at a position close up to the irradiation object to prevent the beam spot size from increasing. In the broad irradiation, on the other hand, the ridge filter 42 needs to be disposed at a position away from the irradiation object because the particle beam necessarily has a largely increased energy width. In the particle beam therapy system according to Embodiment 1, all of the scanning irradiation use vacuum duct 22 and its accompanying parts disposed downstream the scanning electromagnets 25 are dismounted and retracted away from the beam line 100 to ensure a long space.

In FIG. 2, the scanning irradiation use vacuum duct 22 is detachable from the vacuum duct 21 at the flange plane 27 disposed downstream the scanning electromagnets 25. Moreover, when the scanning irradiation use vacuum duct 22 is dismounted from the connection flange, the scanning irradiation use vacuum duct 22 is slid and retracted away from the beam line 100 to a position not to easily overlap with the beam line 100, by the vacuum duct moving mechanism 26 provided with a driving base and a driving rail for supporting the scanning irradiation use vacuum duct 22.

After the scanning irradiation use vacuum duct 22 is dismounted, since the connection flange plane 27 becomes an end plane for the vacuum condition, a beam extraction window 23b is attached to the flange plane 27 as shown in FIG. 3. The ridge filter 42 is lifted up toward the flange plane 27 and placed at a position just beneath a beam extraction window 23b with the ridge filter moving mechanism 261 through the space created by sliding the scanning irradiation use vacuum duct 22 with the vacuum duct moving mechanism 26. At this time, the ridge filter 42 is exchanged from that for scanning irradiation use to that for broad irradiation use. Moreover, the range shifter 43 is moved up and down, if needed, for a bolus 44 and a patient collimator 45 to be mounted. Furthermore, the scatterer 41, which is retracted from the beam line 100 in the case of scanning irradiation, is moved to the beam line 100. In a broad irradiation using the wobbler method, the particle beam is not necessarily spread by the scatterer 41 but is spread by being scanned by the scanning electromagnets 25 in a circular pattern to perform the irradiation.

In this way, the broad irradiation is enabled. The bolus 44 and the patient collimator 45 can be easily mounted by attaching insertion holders therefor to the bottom face of the range shifter 43 with rails or the like. The ridge filter 42 and the range shifter 43 can be inserted using a linear translation mechanism or a rotational translation mechanism driven by air or a motor. While the scanning irradiation use vacuum duct 22 is slidably retracted in the above configuration, providing a rotatable support mechanism also allows the retraction and the insertion of the vacuum duct 22 to be switched to each other by rotating the support mechanism.

If no vacuum separation plane was provided upstream the scanning irradiation use vacuum duct 22 and the scanning irradiation use vacuum duct 22 communicated to the upstream, retraction of the scanning irradiation use vacuum duct 22 would result in breakage of the vacuum condition throughout the beam lines. In this case, it takes time to increase the degree of vacuum. Hence, the gate valve 28 is preferably disposed immediately upstream the scanning electromagnets 25. The gate valve may also be disposed in a position immediately downstream the scanning electromagnets 25. When the scanning irradiation use vacuum duct 22 is dismounted, closing the gate valve 28 allows influence to the degree of vacuum to be limited to only the downstream of the gate valve 28. At that time, configuring the gate valve 28 to have also a function of a final beam extraction window eliminates the need to attach the beam extraction window 23b anew, thereby reducing time for the switching between the broad irradiation and the scanning irradiation.

As described above, the irradiation nozzle 20 thus allows the particle beam irradiation to be switched between the scanning irradiation and the broad irradiation, as shown in FIGS. 2 and 3.

An irradiation apparatus capable of irradiation by switching between the broad irradiation and the scanning irradiation may be configured to have two irradiation lines: a first irradiation line having an irradiation nozzle for the broad irradiation and a second irradiation line having an irradiation nozzle for the scanning irradiation. Such an irradiation apparatus has already been disclosed in Patent Document 1.

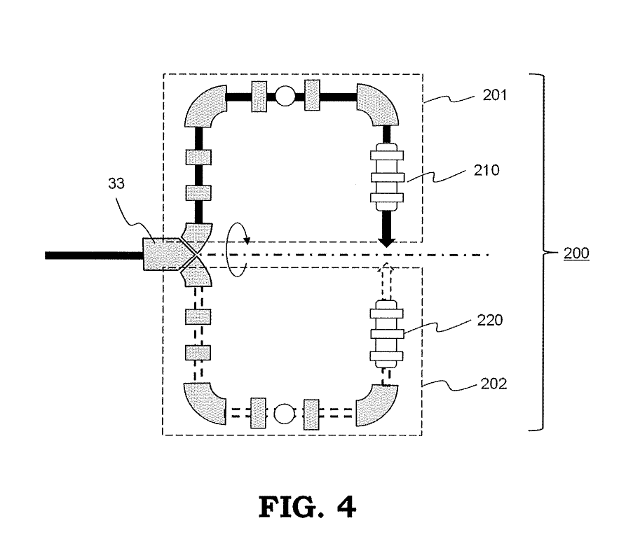

FIG. 4 is a schematic diagram showing an example of an irradiation apparatus having two irradiation lines. The irradiation apparatus, which is designated at 200, is a so-called gantry type irradiation apparatus and is provided in the gantry with two irradiation lines: a first irradiation line 201 and a second irradiation line 202. Delivery of the particle beam is switched between the first irradiation line 201 and the second irradiation line 202 by a delivery line switching device 33. When delivery of the particle beam is switched to the first irradiation line 201, the irradiation is performed from a first irradiation nozzle 210 provided to the first irradiation line. When delivery of the particle beam is switched to the second irradiation line 202, the irradiation is performed from a second irradiation nozzle 220 provided to the second irradiation line.

Here, the first irradiation nozzle 210 is mounted with broad irradiation use parts, and the second irradiation nozzle 220 is mounted with scanning irradiation use parts. The broad irradiation is performed when the delivery line is switched to the first irradiation line, and the scanning irradiation is performed when the delivery line is switched to the second irradiation line. When the irradiation line is switched to the first irradiation line or to the second irradiation line, no rotation of the gantry allows the broad irradiation and the scanning irradiation to be performed respectively from directions different by 180 degrees. In addition, when the irradiation line is switched, for example, from the first irradiation line to the second irradiation line, rotation of the gantry by 180 degrees allows the broad irradiation and the scanning irradiation to be performed from the same direction.

As described above, the broad irradiation and the scanning irradiation can be performed for one and the same patient, using an irradiation nozzle such as the irradiation nozzle 20, shown in FIGS. 2 and 3, configured to be able to perform the broad irradiation and the scanning irradiation by switching parts provided to the irradiation nozzle, or the irradiation apparatus 200, shown in FIG. 4, configured to be able to perform the broad irradiation and the scanning irradiation by switching between the first irradiation line 201 provided with the first irradiation nozzle 210 for the broad irradiation and the second irradiation line 202 provided with the second irradiation nozzle 220 for the broad irradiation.

While a dose monitor is provided in the irradiation nozzle to measure an irradiation dose of the particle beam during both scanning irradiation and broad irradiation, the way of controlling the dose is different between the scanning irradiation and the broad irradiation. The scanning irradiation is performed by controlling particle beam irradiation doses imparted to respective irradiation points in a scanning irradiation region while shifting the particle beam, as with, for example, a spot scanning irradiation method in which irradiation is performed by repeating stop and shift of the particle beam. While the broad irradiation is performed such as using the wobbler method in which the particle beam is spread by being scanned in a circular pattern or using a scattering method in which the particle beam is spread by being not shifted but scattered with a scatterer, either broad irradiation method does not control irradiation doses imparted to respective irradiation points in a broad irradiation region but controls the overall irradiation dose imparted to the entire broad irradiation region.

Next, a method of formulating a treatment plan is described, in which a target irradiation dose distribution is formed by imparting the sum of a broad irradiation dose and a scanning irradiation dose to the same irradiation object, i.e., a diseased site using the irradiation apparatus capable of switching between the scanning irradiation and the broad irradiation described above.

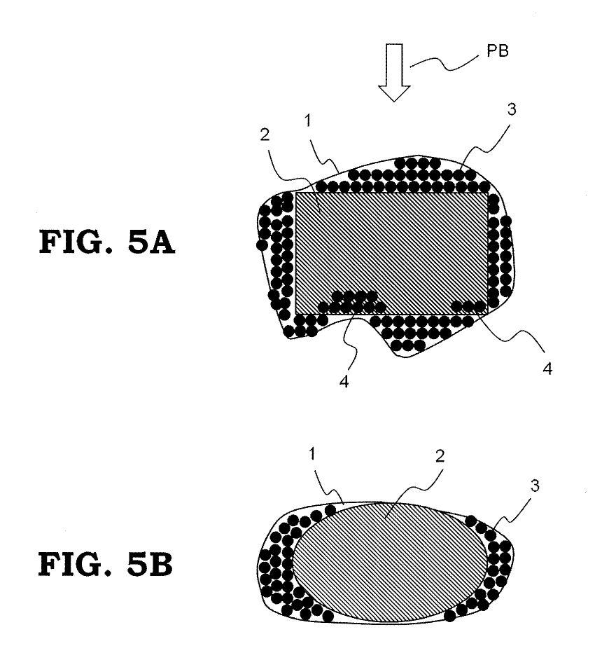

FIGS. 5A and 5B show schematic illustrations of an irradiation based on a treatment plan formulated by the treatment planning apparatus according to Embodiment 1 of the present invention. FIG. 5A is a cross-sectional view of a diseased site, i.e., an irradiation region along the irradiation center axis of the particle beam PB; and FIG. 5B is a cross-sectional view orthogonal to the irradiation center axis. First, a region to be subject to the broad irradiation is set as a broad irradiation region 2 in the diseased site, i.e., an irradiation region 1. And then, a region, other than the broad irradiation region 2, to be subject to the scanning irradiation is set as a scanning irradiation region 3 in the irradiation region 1. In Embodiment 1, the broad irradiation is performed without using the bolus 44. Performing the broad irradiation only using the ridge filter 42, or the ridge filter 42 and the range shifter 43 without using the bolus 44 forms a broad irradiation region having a flat distal end and a flat proximal end as shown by, for example, the broad irradiation region 2 of FIG. 5A. Controlling lateral range of the particle beam, for example, by the patient collimator 45 and by the scanning using the wobbler method allows the cross-section of the broad irradiation region 2 shown in FIG. 5B to be formed in various shapes. Thus, the broad irradiation region 2 is formed in a pillar shape.

For example, the broad irradiation region 2 is set so as to be inscribed in the irradiation region 1. Briefly explaining, since this setting leaves in the irradiation region 1 an unirradiated region other than the broad irradiation region 2, the region needs to be set as the scanning irradiation region 3 to be irradiated by the scanning irradiation. More strictly speaking, in the region to which an irradiation dose is imparted by the broad irradiation, there partially exist portions whose doses are unreached to their respective target irradiation doses. In any case, a target irradiation dose distribution Ds(x, y, z) to be formed by the scanning irradiation can be calculated by subtracting a target irradiation dose distribution Db formed in the broad irradiation region by the broad irradiation from the target irradiation dose distribution D in the entire irradiation region, as expressed by the following Eq. (1):

Dₛ(x,y,z)=D(x,y,z)−D_{b}(x,y,z) (1).

As a result, the irradiation region 1 can be divided into (1) a region irradiated only by the broad irradiation, (2) regions irradiated only by the scanning irradiation, and (3) regions (indicated by irradiation regions 4 shown in FIG. 5A) irradiated by both broad irradiation and scanning irradiation.

Formulation of a treatment plan based on a conventional scanning irradiation needs to solve an optimization problem to determine an irradiation angle, an irradiation dose for each irradiation point, further a scanning path (scanning trajectory) connecting each irradiation point, and the like to form the target irradiation dose distribution D in the entire irradiation region 1. A treatment plan formulated by the treatment planning apparatus according to the present invention only needs to solve an optimization problem for the target scanning irradiation dose distribution Ds calculated from the Eq. (1), thus reducing calculation time for the optimization. In addition, a conventional optimization technique (calculation algorism) can be used, as a matter of course, to form the target scanning irradiation dose distribution Ds.

As described before, the irradiation dose imparted by the broad irradiation is a so-called “fixed irradiation dose”, and shortages of doses to be imparted by the scanning irradiation is “unfixed irradiation doses”. Accordingly, the optimization is, in principle, to approximate the unfixed irradiation doses to the target irradiation doses as close as possible.

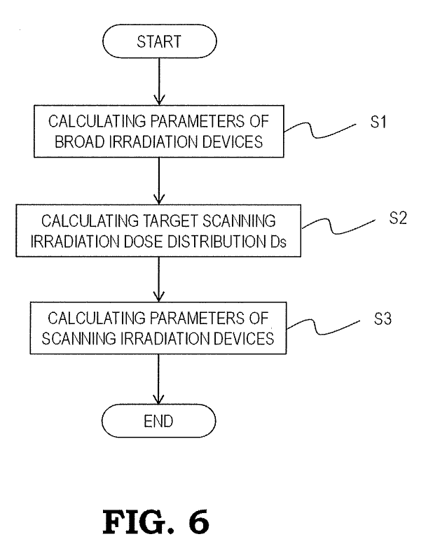

FIG. 6 shows each of operation steps, i.e., an operation flow of the treatment planning apparatus. First, the broad irradiation parameter calculation unit 12 calculates the parameters for the respective devices relating to the broad irradiation in Step S1 so that an irradiation dose imparted to every point by the broad irradiation does not exceed the target irradiation dose, in consideration that the broad irradiation region 2 is inscribed in the irradiation region 1 and the scanning irradiation further imparts irradiation doses to the points in the broad irradiation region 2. Next, the scanning irradiation parameter calculation unit 13 calculates the target scanning irradiation dose distribution Ds (x, y, z) according to Eq. (1) in Step S2. The scanning irradiation parameter calculation unit 13 further solves an optimization problem for the target irradiation dose distribution Ds to calculate parameters for the respective devices relating to the scanning irradiation in Step S3. These calculated parameters for the respective devices are sent to the overall device management apparatus 14.

When starting treatment for a patient, i.e., starting the particle beam irradiation, the irradiation nozzle is, for example, first mounted with the parts for the broad irradiation, and then the respective devices are operated in accordance with their broad irradiation parameters sent from the overall device management apparatus 14, so that the broad irradiation dose distribution Db is formed in the diseased site. After that, the irradiation nozzle is mounted with the parts for the scanning irradiation, and then the respective devices are operated in accordance with their scanning irradiation parameters sent from the overall device management apparatus 14, so that the scanning irradiation dose distribution Ds is formed in the diseased site. By both irradiations, the irradiation dose distribution D=Db+Ds is imparted to the diseased site. Since the irradiation dose distribution only needs to satisfy D=Db+Ds, it is no matter which the broad irradiation or the scanning irradiation is performed first in order.

As described above, the treatment planning apparatus 10 formulates a treatment plan for both broad irradiation and scanning irradiation to form a target irradiation dose distribution in a diseased site, and a particle beam irradiation is performed in accordance with the treatment plan. This brings about the following effects. The broad irradiation is a conventionally used irradiation method and has a merit in that there are many actual results in clinical practice; however, since broad irradiation regions are difficult to conform respectively to various shape diseased sites, a bolus is necessary for a broad irradiation region to conform to a diseased site shape on a patient-by-patient basis. In contrast to that, the scanning irradiation is capable of forming various irradiation regions and various irradiation dose distributions by controlling the parameters for the respective devices; however, optimization calculation for the scanning irradiation takes time in formulating a treatment plan. According to Embodiment 1 of the present invention, the broad irradiation imparts an irradiation dose to a large portion of an irradiation region and the scanning irradiation imparts irradiation doses to irradiation points in the remaining portions, which are mainly peripheral portions. This reduces the scanning irradiation regions thereby reducing time to formulate a treatment plan, and brings about an effect of being able to imparting irradiation doses to a diseased site having various shapes with high accuracy. Furthermore, the scanning irradiation can be performed in a short time, thus facilitating performing the scanning irradiation, for example, with so-called respiration synchronizing control, i.e., during a less movement phase of a diseased site in the respiration cycles. Performing the scanning irradiation with the respiration synchronizing control brings about an effect of being able to impart irradiation doses with higher accuracy.

### Embodiment 2

In Embodiment 1, no bolus is used in the broad irradiation. A bolus is usually fabricated for the range of the particle beam to conform to the shape of a diseased site, i.e., fabricated to adjust the energy distribution of the particle beam to the range conforming to a lower portion shape (distal shape) of a diseased site. A broad irradiation using a bolus allows for forming an irradiation dose distribution in conformity to a distal shape of a diseased site. However, irradiation from one direction is difficult to form an irradiation dose distribution in conformity to both distal and proximal shapes of a diseased site, i.e., the shape of the entire diseased site. Hence, a target irradiation dose distribution has been formed in the entire diseased site by a so-called multi-port irradiation such that irradiations are performed from, for example, an upper direction using a bolus for the distal shape and from the lower direction using a bolus for the proximal shape.

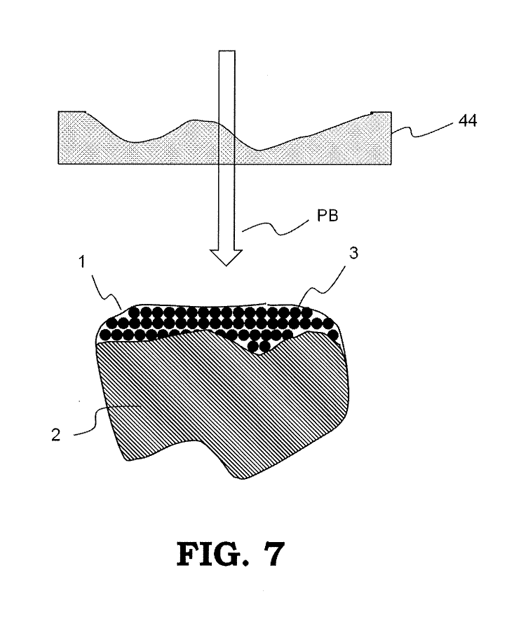

In Embodiment 2, the broad irradiation is performed using a bolus, for example, a bolus for distal shape that forms an irradiation dose distribution in an irradiation region whose shape is the same as a distal shape of only a portion of an irradiation object, and the scanning irradiation is performed to impart irradiation doses to a portion to which the broad irradiation cannot impart an irradiation dose. A schematic illustration of the irradiations is shown in FIG. 7. As shown in FIG. 7, a bolus 44 is used to form a broad irradiation region 2 conforming to the shape of a portion opposite to the incident side of the particle beam PB, i.e., conforming to the distal shape of an irradiation region 1. However, the broad irradiation using the bolus 44 alone cannot extend the broad irradiation region 2 to an irradiation region including a region conforming to the proximal shape of the diseased site. For that reason, the region in which the broad irradiation cannot form an irradiation dose distribution is regarded as a scanning irradiation region 3, and the scanning irradiation imparts irradiation doses to the region.

In this case, it is sufficient to form an irradiation dose distribution only in the region proximal to the incident side of the particle beam by the scanning irradiation, thus allowing a treatment plan for the scanning irradiation to be formulated in a shorter time than Embodiment 1. Moreover, each irradiation of the scanning irradiation can be performed in a short time, thus facilitating performing the scanning irradiation, for example, in synchronism with respiration. Performing the scanning irradiation in synchronism with respiration brings about an effect of being able to impart irradiation doses with higher accuracy.

### Embodiment 3

In Embodiment 2, the broad irradiation is performed using a bolus conforming to a distal shape of a portion of a diseased site. Conventionally, a bolus has been fabricated, each time on a patient-by-patient basis, as a patient-specific bolus conforming to the diseased site of a patient. Embodiment 3 is characterized in that the broad irradiation is performed not using a patient-specific bolus but using a bolus that is selected to approximate to a distal shape of a diseased site among a plurality of device-specific different shape boluses prepared beforehand. The plurality of different shaped boluses is referred here to as versatile boluses.

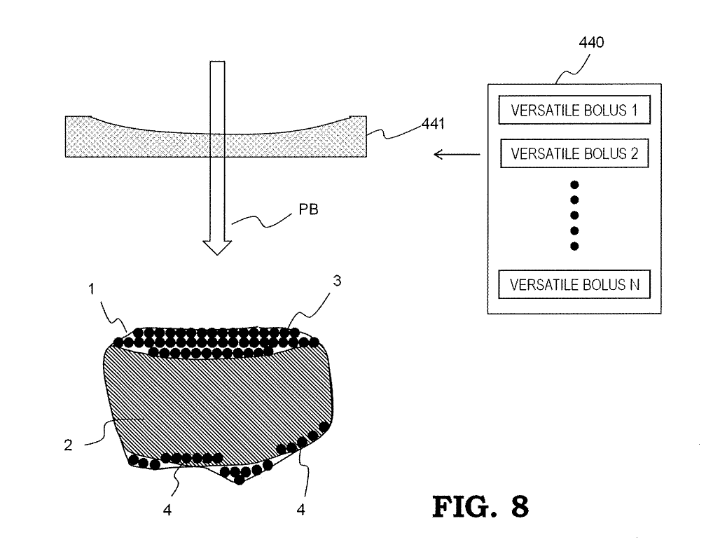

FIG. 8 shows a schematic illustration of irradiation performed according to Embodiment 3 using a versatile bolus. For example, one versatile bolus 441 to be mounted to the irradiation nozzle for use in the broad irradiation is selected among N versatile boluses stored in a versatile bolus box 440. In a case of using one versatile bolus among the N versatile boluses beforehand prepared independently on the shape of a diseased site, the broad irradiation region is difficult to conform to the distal shape of the diseased site. A versatile bolus that forms the broad irradiation region 2 to be inscribed in the distal shape of a diseased site is preferably selected as the versatile bolus 441 among the plurality of versatile boluses. The broad irradiation is performed using the selected versatile bolus. However, by the selected versatile bolus 441 alone, an irradiation dose cannot be imparted to the entire irradiation region 1. For that reason, the regions in which the broad irradiation cannot form a target irradiation dose distribution are regarded as the scanning irradiation regions 3 and the irradiation region 4, and the scanning irradiation imparts irradiation doses to the regions.

This reduces the scanning irradiation region 3 compared to Embodiment 1 that performs the broad irradiation without using a bolus, thus allowing a treatment plan for the scanning irradiation to be formulated in a further shorter time than Embodiment 1. Moreover, each irradiation of the scanning irradiation can be performed in a short time, thus facilitating performing the scanning irradiation, for example, in synchronism with respiration. Performing the scanning irradiation in synchronism with respiration brings about an effect of being able to impart an irradiation dose with higher accuracy.

### NUMERAL REFERENCE

1: irradiation region; 2: broad irradiation region;
3: scanning irradiation region: 10: treatment planning apparatus;
11: overall data management unit;
12: broad irradiation parameter calculation unit;
13: scanning irradiation parameter calculation unit;
20: irradiation nozzle; 210: first irradiation nozzle;
220: second irradiation nozzle; 30: accelerator; 44: bolus; and
441: versatile bolus.

1. A treatment planning apparatus configured to formulate a treatment plan that allows a particle beam therapy system to irradiate an irradiation object with a particle beam extracted from an accelerator, by switching between a scanning irradiation from a scanning irradiation nozzle mounted with scanning irradiation use parts for the scanning irradiation that is performed while shifting the particle beam and controlling an irradiation dose imparted to each of points in the irradiation object and a broad irradiation from a broad irradiation nozzle mounted with broad irradiation use parts for the broad irradiation that is performed by controlling a total irradiation dose imparted to a region in the irradiation object, the treatment planning apparatus comprising:
an overall data management unit configured to store a target irradiation dose distribution to be formed in the irradiation object;
a broad irradiation parameter calculation unit configured to calculate operational parameters for respective devices, such as the accelerator and the broad irradiation nozzle, to operate during the broad irradiation; and
a scanning irradiation parameter calculation unit configured to calculate operational parameters for the respective devices, such as the accelerator and the scanning irradiation nozzle, to operate during the scanning irradiation,
wherein the broad irradiation parameter calculation unit and the scanning irradiation parameter calculation unit cooperatively calculate and determine the operational parameters for the respective devices, such as the accelerator and the broad irradiation nozzle, to operate during the broad irradiation and the operational parameters for the respective devices, such as the accelerator and the scanning irradiation nozzle, to operate during the scanning irradiation, so that the sum of irradiation doses imparted by both broad irradiation and scanning irradiation forms the target irradiation dose distribution.

2. The treatment planning apparatus of claim 1, wherein the broad irradiation is performed without using a bolus.

3. The treatment planning apparatus of claim 1, wherein the broad irradiation is performed using a bolus for forming an irradiation dose distribution in an irradiation region whose shape is different from a distal shape of the entire irradiation object.

4. The treatment planning apparatus of claim 3, wherein the bolus forms an irradiation dose distribution in an irradiation region whose shape is the same as a distal shape of only a portion of the irradiation object.

5. The treatment planning apparatus of claim 3, wherein the broad irradiation use parts includes a plurality of device-specific boluses prepared beforehand, and the bolus is selected among the plurality of boluses.

6. A particle beam therapy system capable of irradiating an irradiation object with a particle beam extracted from an accelerator by switching between a scanning irradiation from a scanning irradiation nozzle mounted with scanning irradiation use parts for the scanning irradiation that is performed while controlling an irradiation dose imparted to each of points in the irradiation object and a broad irradiation from a broad irradiation nozzle mounted with broad irradiation use parts for the broad irradiation that is performed by controlling a total irradiation dose imparted to a region in the irradiation object, wherein respective devices, such as the accelerator, the scanning irradiation nozzle, and the broad irradiation nozzle, are controlled to operate in accordance with operational parameters determined for the respective devices by the treatment planning apparatus of claim 1.
